(19) United States
(12) Patent Application Publication
(10) Pub. No.: US 2005/0047997 A1
(43) Pub. Date: Mar. 3, 2005

(54) Method for the evaluation of the efficiency of a therapeutic action upon the human organism

(76) Inventor: Vladimir Borisovich Grinevich, Saint-Petersburg (RU)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 10/755,563

(22) Filed: Jan. 12, 2004

Related U.S. Application Data
(63) Continuation-in-part of application No. 09/462,590, filed on Jan. 7, 2000, now abandoned.

(51) Int. Cl. G06F 17/60; A61K 49/00

(52) U.S. Cl. 424/9.1; 424/9.2; 705/3

(57) ABSTRACT

The present invention pertains to the field of medicine and more precisely relates to a method for diagnosing the action of medical preparations as well as medicating, psycho-corrective, mechanical and physiotherapeutic actions applied on the organism of a patient. This invention may be used in the field of pharmacy as well as in the practice in therapy institutes. After a preliminary study, the method of the present invention comprises selecting a research group and monitoring a group including patients of the same sex and approximately the same age. The patients are applied the therapy to be studied and a monitoring is carried out before drawing the conclusions about efficiency according to the treatment of the disease or the recovery and/or the prevention of a pathological symptom occurrence. The groups of subjects are divided by a number that ensures statistical certainty, with a period similar in each group starting from a disease relapse (aggravation) to the beginning of the patient's participation to the clinical experiment, and with an identical location of the pathological process. This method is characterised by an identical homeostatic condition especially concerning lipids. The therapeutic action is monitored for a time period which is calculated individually according to the selected groups. The therapeutic action efficiency is determined from the duration necessary for treating or curing the disease and/or preventing the occurrence of a pathological symptom and its effect on the homeostasis. The efficiency of the therapeutic action is also determined from its effects on the homeostasis in the case, for example, when the efficiency of said action depends on the homeostasis characteristics. The index discrepancies relative to average statistical values are estimated for the corresponding groups both when increasing or decreasing in the case of a given disease.

This invention is related to medicine, particularly, to the evaluation of the therapeutic action of medical preparations, medications, as well as psycho-adjusting, instrumental and physiotherateutic treatment upon the human organism, and can be applied in pharmaceutics and in medical practice.

### DESCRIPTION OF THE PRIOR ART

The proposed method permits to evaluate therapeutic or other effects upon the human organism and to make a qualitative analysis of the action suggested for treatment based on the objective quantitative indicators that characterize the patient's state. “Method for the Efficiency Evaluation of the Treatment of Infectious and Bodily Diseases” is known (Author's Certificate 1814070, Al, SU, published 07. May 93, No. 17, GO1N 33/48), which is essentially based on introducing a pathogen into the human organism and measuring the calibration curve that reflects dependence between the number of microbes and their optical density, thus determining the number of microbes that were blocked. This method permits to evaluate quantitatively the therapeutic effect of treatment upon the human organism. However, the method evaluates the therapeutic efficiency only by humoral and cellular factors and lacks an integral approach to the evaluation.

“Method for the Evaluation of the Therapeutic Action of Medical Preparations upon Water-Salt and Acid-Alkali Balance” is known (Author's Certificate 1802318, Al, SU, published 15. Mar. 93, No. 17, GO1N 33/48). According to this method, the tissue sections of internal organs are made, the total number of chief cells and inserted cells is counted, thus the size of the basal part of each cell is determined. The conclusion on the state of the whole organism is made based on the study of cellular changes. The given method permits to evaluate the therapeutic effect upon the tissues of internal organs, with the dynamic processes taken into consideration. However, a simple transfer of observed ratios between the specific characteristics to the entire general combination of characteristics may produce a large scatter in conclusions relative to the state of the organism. Moreover, this evaluation method requires invasion into the human organism, which is undesirable. Finally, the therapeutic effect of new medical preparations is evaluated by only two characteristics that cannot fully reflect the state of the organism as a whole. So, the given method does not provide the required accuracy of the efficiency evaluation of medical preparations.

“Method for the Evaluation of the Toxicity of Physical and Chemical Actions” (93019287, A, RU, published 10. Feb. 97, No. 4, GO1N 33/48) is known. The method is based on testing the cells of buccal epithelium and measuring the changes in the diameter of cellular nuclei. This method permits to extrapolate the data characterizing the therapeutic effect upon the human organism with the patient's individual sensitivity taken into consideration. However, the reactivity of the buccal cells does not permit to evaluate the specificity of the effect produced upon the organism and to develop a universal method for measuring the reaction of a biological object to the studied action. The above method itself is not universal either, since the sensitivity threshold of buccal cells is strictly individual. This fact makes it impossible to conduct a correct comparative analysis of the therapeutic effect by the threshold level, since this level is different for the studied groups. Besides, the discussed method does not permit to specify the indicators that characterize the studied therapeutic effect. Moreover, this method allows to determine only negative therapeutic effects giving no opportunity to evaluate positive ones, which decreases the accuracy of the overall efficiency evaluation. Though the method in question permits to analyze the sensitivity of the organism to industrial and ecological factors, the scope of such factors is very limited.

“Method for the Efficiency Evaluation of Medical Preparation for Treating Gastric Ulcer in Exacerbation Stage” (93047427, A, SU, No. 8, 1996) is the closest to the proposed invention by its technical essence. According to this method, a group of patients with a comparable initial surface area of ulcerous defect and a similar localization of the defect in the middle third of the stomach is formed. The patients in the group are prescribed the medical preparation, which has to be evaluated. The average rate of healing is measured and compared with the standard cicatrization rate. The above method uses such an integrating characteristic as the surface area of ulcerous defect. This characteristic, however, relates only to ulcers. The method permits to measure such statistically significant indicators as the difference in temporal characteristics of various processes for each experimental group of patients, as well as to establish the correlation of this difference with the action of the studied medical preparation. The above method, however, leaves out of consideration the cause of such a factor as the ability of the mucous membrane to serve as a very sensitive indicator of pathological processes that occur at the level of the entire organism, since the relationship of integral characteristics of the mucous membrane and its structural modification, on the one hand, and the changes in the homeostasis of the organism, on the other, is ignored. The method also ignores the plurality of indicators that characterize the internal medium of the organism. Therefore, it is difficult to interpret the results of the study and, above all, to rank the medical preparations by their therapeutic effect upon the progress of healing. It is also impossible to make a sufficiently full evaluation of the therapeutic action of medical preparations and other types of medical procedures such as psycho-adjusting, instrumental and physiotherapeutic. Finally, the method does not provide for the analysis of factors that are identical and universal for different types of diseases.

Another method for the evaluation of the therapeutic efficiency, which is very close to the proposed invention by its technical essence, is the one described in the article “Ulcer Exacerbation After Remission Achieved Through the Use of Omepazol and Cytametidine” (Scandinavian Journal of Gastroenterology, special edition, 31. Jan.-02. Feb. 1990, published by UNIVERSITETSFORLAGET, Oslo, Russian translation, p. 10). According to this method, groups of patients are formed by comparable criteria such as the type of disease. The studies were performed on two groups of patients with, respectively, prepyloric ulcers and ulcers of the stomach body that were comparable in characteristics. The patients were monitored during the ulcer healing stage and for the following six months. After the exclusion criteria were applied, the number of patients participating in the experiment amounted to not less than 217 persons in both experimental groups. The medical preparations to be studied were prescribed to the patients, with the therapeutic effect monitored by a double blind method. The groups consisted of patients aged 18 through 80 years. At the first stage of the experiment the patients' state was controlled after 4 and 6 weeks, at the second stage—after 2, 4, and 6 months. The control was based on the clinical indicators obtained by endoscopy and was performed in strict conformity with the schedule. The control point at the first stage was fixed in 4 weeks, which is the general duration of ulcer healing. However, the specifics of the sex and age composition of the compared groups, as well as the individual characteristics of patients comprising these groups, for instance, a larger number of smokers in one of the groups, were left out of consideration. When forming the groups, the complex indicators characterizing the general state of the patients' organisms, for example, the reactivity in combination with homeostasis characteristics that reflect the in-depth processes in the organism, were also ignored. The above method permitted to partially subdivide the efficiency estimates of the therapeutic effect depending on the localization and type of ulcerous defect, but could not provide for the ranking of action depending on its therapeutic effect or the ranking of medical preparations. This method permits to evaluate the action efficiency of one medical preparation only in comparison with another. This fact decreases the accuracy of the evaluation by making it dependent on the systematic error inherent to the results of the examination of the reference group. Besides, this evaluation method permits to evaluate only the effect of medicinal action relative to one specific type of disease, leaving out of consideration the state of homeostasis of the entire organism. The criteria for including patients into reference groups are widely scattered, for example, by age, which decreases the accuracy of the evaluation of therapeutic action efficiency. If the action produced a positive effect faster as compared with the usual healing duration, the given method does not permit to accurately specify the time when the positive effect occurs, since the method of control is traumatic and cannot be performed at any time. The patients were not definitely differentiated by sex, age, harmful habits and the duration of the disease. The requirements imposed on the accuracy of determining the duration of healing period were met only through the use of organizational procedures. The patients whose interval between the control point and the real healing exceeded the preset value were left in the experiment, because in general the exclusion of patients from an experiment, decreases the number of cases used as a basis for evaluating the action efficiency, thus decreasing the accuracy of this evaluation. This is confirmed by the fact that the results of treatment after 4 and 6 weeks were almost identical. The above method makes no differentiation in the efficiency of medical treatment for those patients who show no clinical signs. So, the discussed method for the evaluation of the efficiency of therapeutic action is not universal; it does not provide for a differentiated evaluation of medical preparations, nor does it increase the evaluation accuracy. A full healing of ulcer is a primary criterion for evaluating the efficiency of the action.

### DISCLOSURE OF THE INVENTION

The main goal of the present invention is to create a differentiated method for the evaluation of the efficiency of therapeutic action upon the human organism that. permits to make a comparative study of therapeutic means, rank them by their efficiency, and determine the most efficient among them. The method makes it possible to specify the indications for the application of the above means for cases of different localization of pathological processes by taking into consideration, among other things, the whole spectrum of the accompanying pathology that characterizes the entire organism. The method also permits to specify the indications for applying therapeutic means with the individual specifics of the patient taken into consideration.

The above goal is achieved in the proposed method for the evaluation of the efficiency of therapeutic action upon the human organism, wherein the patients are preliminarily examined and assigned to either experimental or reference group consisting of persons of comparable sex and age. They are prescribed the therapeutic action to be evaluated. The resulting therapeutic effect is then measured and conclusions as to the efficiency of this action are made based on the fact of healing or the elimination of pathological signs and/or the prevention of occurrence of pathological signs.

The proposed method is distinguished by the number of patients in the studied groups that provides the statistical reliability, besides the patients of the same group are characterized by a comparable duration of the period elapsed from the beginning of exacerbation or from the start of the disease till the patient is included into the clinical experiment.

The patients of the same group have the same localization of the pathological process with a similar state of homeostasis, mainly lipid. The therapeutic effect is monitored in periods set individually for each of the groups participating in the experiment. The efficiency of the therapeutic action is evaluated depending on the duration of the healing period or the period necessary to eliminate and/or prevent the occurrence of a pathological sign, as well as by its effect of the homeostasis.

For example, the efficiency of therapeutic action upon homeostasis can be evaluated in case when this efficiency, among other things, depends on the characteristics of homeostasis that cause either positive, or negative deviations of indicators from the average statistical values for the corresponding groups of patients suffering a specific disease. When the efficiency of the therapeutic action is evaluated for the cases of ulcer, the patients included into the groups are characterized by a similar duration of the acute or stationary phase from its relapse (from the moment of ulcer opening) up to the moment the patient was included into the clinical experiment. The patients in the groups have a comparable initial surface area and localization of ulcerous defects. The efficiency of the therapeutic action is evaluated by its effect on homeostasis. The evaluation process takes into consideration not only the duration of ulcer cicatrization and the time necessary for the prevention of complication, but, among other factors, the characteristics of homeostasis that feature both positive and negative deviations from the average statistical values for the corresponding group of patients suffering from the given disease.

The proposed method for efficiency evaluation permits to develop a universal technique for evaluating the efficiency of therapeutic action with enhanced accuracy. Besides, the method provides for a differentiated evaluation of the efficiency, simplifies, in certain cases, the evaluation procedure, and decreases the traumaticity of the examination. The therapeutic action evaluated can be medicinal, instrumental, or psycho-adjusting. During the experiment the reactivity of the organism is studied. Such evaluation of the efficiency of therapeutic action reliably establishes the fact of healing or the elimination and/or prevention of occurrence of a pathological sign and gives the assessment of the level (degree) of the effect.

### BRIEF DESCRIPTION OF DRAWINGS

FIG. 1a—the table of results obtained in the studied group for a number of ulcer patients treated with “Maalox” medication;

FIG. 1b—the continued table of results obtained in the studied group for a number of ulcer patients treated with “Maalox” medication;

FIG. 2a—the table of results obtained in the reference group for a number of ulcer patients treated with “Zerocid” medication;

FIG. 2b—the continued table of results obtained in the reference group for a number of ulcer patients treated with “Zerocid” medication;

FIG.3—the table of results obtained in the studied and reference groups for a number of ulcer patients treated with “Corinfar” and “Prestarium” medications;

FIG.4—the plot of ulcerous defect surface area S (sq.centimeters) versus time (days).

### VARIANT OF THE INVENTION EMBODIMENT

The gastroduodenal mucous membrane is known to be a sort of indicator that readily reacts to pathological processes in the organism.

For example, for patients with ulcerous defects in the gastroduodenal mucous membrane the index of dyslipoproteidemy (DLP) at the stage of chronic pancreatitis, which often precedes the ulcer, plays a certain role in the disease pathogenesis. The role of such a complex index as DLP in the pathogenesis of diseases of digestive organs, primarily lever, pancreas, and stomach is very important. The role of the complex index DLP in forming various diseases of the organism is widely known. There exists a certain relation of the DLP index with the specifics of the clinical course of the disease with those patients who go through a relapse of the disease of digestive organs. Based on the above mentioned, the proposed method for the evaluation of the efficiency of therapeutic action upon the human organism is implemented as follows:

### EXAMPLE 1

Two groups of patients, experimental and reference ones, are formed based on comparable sex and age characteristics. As a result, each group consists of patients of the same sex or features the same proportion of males and females who differ in age by not more than one year. After a preliminary examination, the patients are ranked by a comparable localization of ulcerous defects. Thus, patients with the stomach ulcer are ranked by the ulcerous defect localization with the accuracy of 1/9th of the stomach sections: cardia, subcardia, upper, middle and lower thirds of the stomach body, angle, anthral and prepyloric sections, pylorus. The initial surface area of the ulcerous defect is measured. Then the current stage (phase) of the disease: acute stage (the stage of ulcer forming), stationary stage or “plateau” and the stage of reparation proper (see FIG. 4) is determined for each patient on the basis of clinical, laboratory and instrumental indications. These phases characterize the period of time that passed since the beginning of relapse (exacerbation or start) of the disease till the patient was included into the clinical experiment. The method in question uses homeostasis indications, particularly those related to lipid metabolism, for the evaluation of the efficiency of the therapeutic action. Based on the above principle, the patients are divided into subgroups according to the following classification:
cholesterol (CS);
high density lipoproteins (HDLP);
low density lipoproteins (LDLP);
very low density lipoproteins (VLDLP);
triglycerides (TG);
atherogenicity factor (AF).

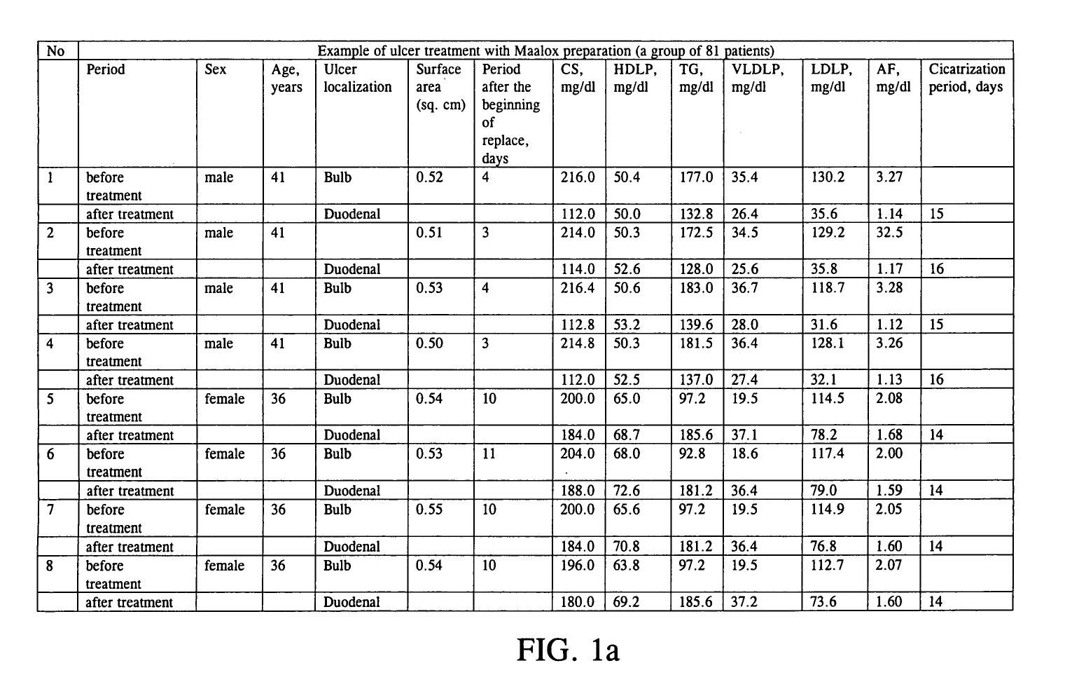

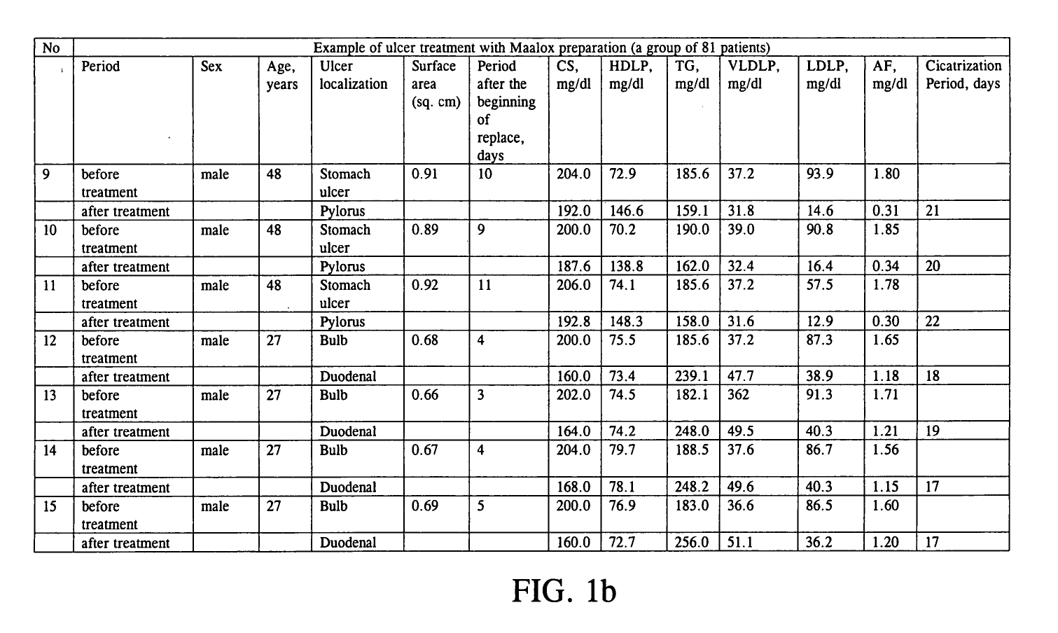

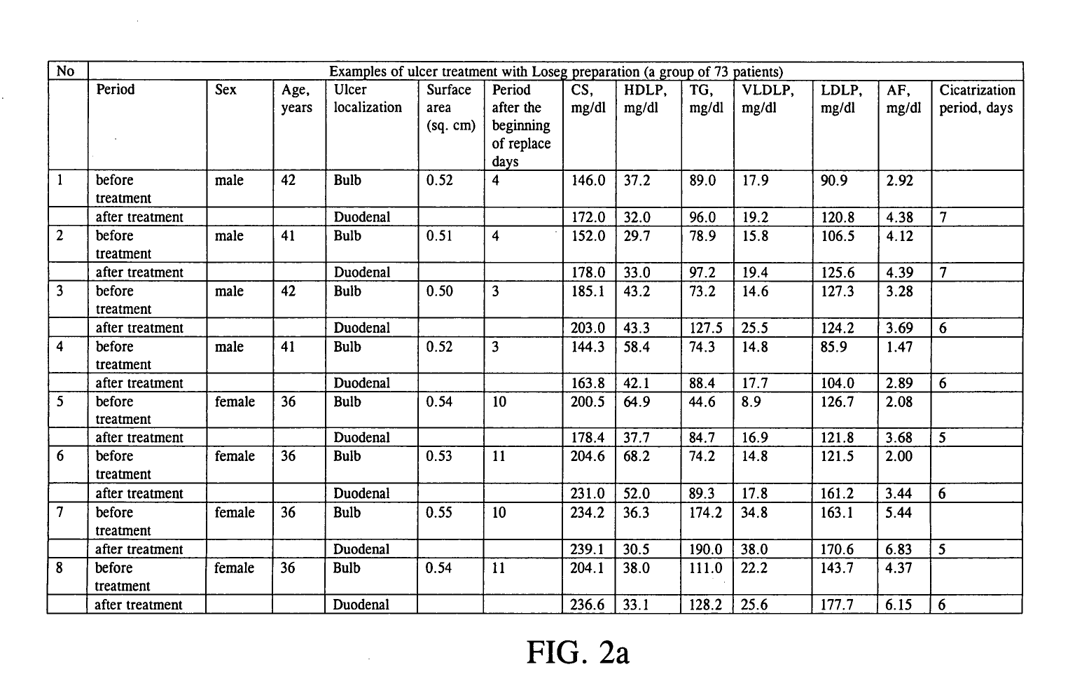

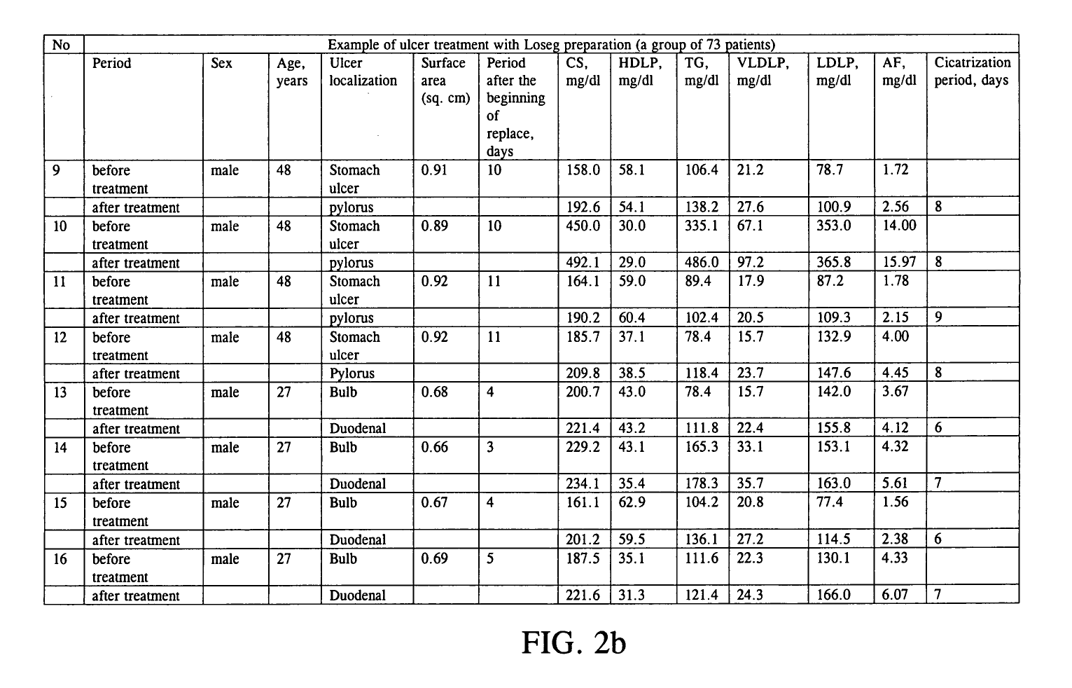

Thus, the patients are taken at the same phase of disease relapse and at different phases of stationary ulcer development (see FIG. 1a, b; lines from 5 to 11; FIG. 2a, b; lines from 5 to 12). When monitoring the complex of the specific characteristics of homeostasis, this technique, however, takes into consideration individual features. The table shows the following localization:
duodenal bulb (see FIG. 1a, lines from 1 to 8; FIG. 1b, lines from 12 to 15; FIG. 2a, lines from 1 to 8; FIG. 2b, lines from 13 to 16);
gastric ulcer (GA), pylorus (see FIG. 1b, lines from 9 to 11; FIG. 2b, lines from 9 to 12).

The quantitative number of patients in groups must provide the statistical reliability of the results. In the given example the group treated with a reference medical preparation consisted of 73 patients, while the group treated with the medical preparation that had to be evaluated consisted of 81 patients. The reference group was treated with a medication belonging to proton pump blocking agent (for example, Zerocid), while the experimental group was treated with a medication belonging to antacids (for example, Maalox). If the studied medications are only to be ranked by the efficiency of their action upon the organism rather than compared with each other, placebo can be used as a reference medication. The control time is determined for the selected groups of ulcer patients. The control is performed individually for each ranked experimental group. The ranking is based on the statistical data depending on the phase (stage) of ulcer development:
acute phase (forming stage)—3-4 days after the relapse began;
stationary phase or “plateau” stage—8-40 days;
stage of reparation proper—according to the trend (see FIG. 1a, b; FIG. 2a, b).

The initial efficiency criterion was a complete healing of ulcerous defect combined with the dynamics of the change in homeostasis characteristics. The individual temporal norm was represented by a time interval calculated on the basis of individual characteristics of the ulcer healing process that corresponded to the individual characteristics of the homeostasis indications, for instance, to the characteristics of lipid metabolism calculated by the known formulas. During the treatment a decrease in cholesterol (CS) was observed (see FIG. 1a, b), which served as a positive indicator that the probability, for example, of myocardial infarction was reduced. In the experimental group of patients (see FIG. 1a, b) after the period from 14 to 22 days the level of cholesterol related to high density lipoproteins (HDLP) increased. Being an indication of sanogenesis, this fact testified to the prevention of development or reduction in the probability of such pathological states as, for instance, atherosclerosis, cancer and others. The levels of triglycerides (TG), low density lipoproteins (LDLP) and very low density lipoproteins (VLDLP) decreased, which was a positive sign. The atherogenicity factor (AF) decreased, which indicated that atherosclerosis development decelerated. Having compared the above facts with the average statistical norms, one can make a conclusion about the nature of the therapeutic action. For example, ulcerous defects cicatrize faster (see FIG. 2a, b) when treated with Zerocid, but the changes in the lipid spectrum indications show that it produces a clearly negative effect upon the atherogenesis and oncogenesis in the organism of an ulcer patient. This is confirmed by the fact that the patients in the experimental group (see FIG. 2a, lines 1-16) featured decreasing HDLP indicators and increasing TG and AF indicators.

### EXAMPLE 2

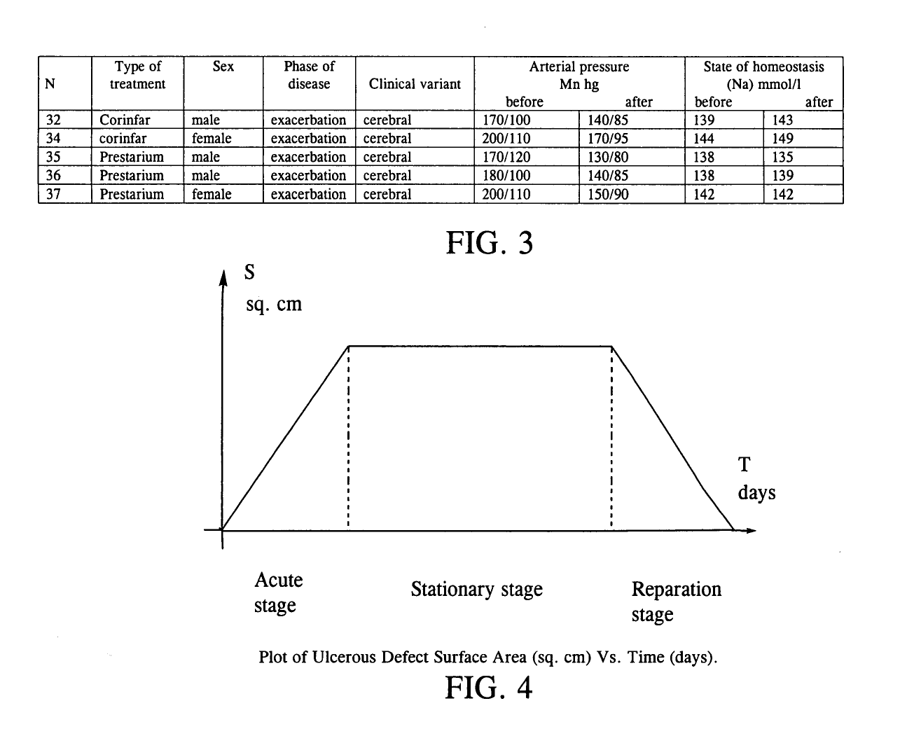

Selected hypertension patients are divided into two groups. The reference group includes 48 patients and the experimental group includes 56 patients. Each group consists of 60% of females, the rest being males. All the patients in the same group differ in age by not more than 1 year. The patients from the experimental group are treated with a hypotensive medication belonging to calcium current blocking agents—Corinfar, (see FIG. ??, lines from 1 to 3), while those from the reference group are treated with a medication that blocks angiotensin-transforming enzyme (ATE) Prestarium (see FIG. 3, lines from 4 to 6). The period from the beginning of the relapse till the inclusion of the patient into the experiment is chosen to be 2 days. The phase of the disease is selected to be hypertension of medium exacerbation. The prevailing disease localization is its cerebral variant, in other words, the variant characterized by prevailing damage of cerebral vessels. The initial level of the homeostasis characteristics is set by the characteristic of electrolytic homeostasis, i.e. the sodium level, and is equal to 140 mmol/l for both groups. The patients' arterial pressure was monitored every other day, which corresponded to the period of time that is characterized by a significant decrease in arterial pressure under the effect of ATE blocking agent. It can be concluded that the arterial pressure decreased by comparable values as a result of treatment by both medications. However, in case when Corinfar was used, the level of sodium increased up to 146 mmol/l, while the treatment with ATE blocking agent Prestarium reduced this level down to 137 mmol/l. The decrease of the sodium level in the latter case is clearly a positive change in the homeostasis of hypertension patients.

### INDUSTRIAL APPLICATION

The method for the evaluation of the therapeutic action upon the human organism can be applied in medical institutions for determining the most effective medicinal, physiotherapeutic or other therapeutic action. The given method can be applied both for comparing different medical preparations and for ranking them when treating different groups of patients. The method permits to evaluate the therapeutic effect of medical preparation produced by various pharmaceutical manufacturers in order to determine their efficiency and safety. This method can also be used for synthesizing new medical preparations.

A question of choice of necessary and sufficient number of characteristics is not solved by the prior art. “The supply of nutrients, the amount of respiratory gases, the removal of wastes, the concentration of specific substances of choice, the acidity, the osmotic pressure, temperature cardiovascular homeostasis, (including the effects of gravity and exercise on blood pressure) are necessary but not necessarily sufficient characteristic for achieving the benefits of the invention, which solves the problem of choice of sufficient characteristic number for univocal homeostasis characteristic that makes it possible to provide accuracy of the evaluation of the efficiency and safety of a therapeutic action upon the human organism. The chosen features of homeostasis in accordance with the invention have a univocal logical link, described by definite logics, between the chosen concrete sufficient characteristics of homeostasis and time interval from the last diseases relapse until a moment the patients are included into a clinical experiment.

The requirement for comparable sex and age composition means that patient efficiency and safety of a therpeautic action are evaluated for specific ages or age groups. All patient assigned to a reference group may be within a predetermined age group, i.e, that is all members of a particular reference group differ in age by no more than a predetermined amount of time (e.g., 5 years, or 1 year). Within any reference group, the sex is the same, age of patients is approximately the same, and the localization of pathological process are similar. Patients are included in group for study with same sex parameter (in one group patients have the same sex—only male or only female). Age difference between patients included in a group preferably do not exceed one year. And for including in group for study the similar localization of pathological process is desirable, e.g., localization of ulcerous defect with accuracy of 1/9-th of the stomach sections: cardia, subcardia, upper, middle and lower thirds of the stomach body, angle, anthral and prepyloric sections, and pylorus.

Results of therapeutic action is determined depending on period of healing together with influence of this period upon homeostasis. Selection of characteristics for description of the homeostasis cited in specification (cholesterol, high density lipoproteins, low density lipoproteins, very low density lipoproteins, triglycerides, atherogenicity factor). They define interference healing ulcerous defect velocity and homeostasis condition, where the homeostasis condition is described by union of above identified characteristics. Efficiency is estimated only on the point of interdependent influence of parameters [1] and [2] one to another. Such generalized estimation nobody had executed before Professor Grinevich. They were satisfied only by estimation of healing ulcerous defect velocity, i.e., only the parameter [1]. However, estimation of only the healing velocity is not enough, since it may occur that, when quick positive result of ulcerous defect healing is reached, other organs could suffer. In this case ulcerous defect is some kind of safety value which allows to act upon the homeostasis of organism on the whole at the expense of acceleration or deceleration of healing ulcerous defect process.

The 3^{rd}, period from the beginning disease till the beginning of study were not anticipated by Scheiman since Scheiman operates with other time period—from the beginning of using of medicine till the beginning of study, which allows to determine injury of organism over the using of this medicine. Under the Scheiman it is possible to estimate only the safety of medicine (injurious effect degree). Under the Grinevich it is estimated simultaneously efficiency of concrete pathological organ healing process (restoration of its functioning) and safety for another organs and whole organism.

Under the Hon it is estimated external action of medicine upon the organism, and Hon states that similar characteristics of homeostasis are achieved by different doses, i.e., by different external action. Under the Grinevich it is estimated stability of organism itself, i.e., equilibration of its internal processes, and these processes allow within the limits of this stability to determine most effective external medicine action. The state-of-the-art methods consider essential only the presence or absence of ulcerous defect in the stomach or duodenum and the degree of mucous membrane legion without due regard for the location of the ulcerous defect and its relation to the homeostasis of patient's organism, characterized by a set of indications including routine blood indications and, which is obligatory, indications of lipid and hydrocarbon types of metabolism. Besides, in previous methods each of the considered “inclusion-exclusion” criteria is analyzed separately, rather than within the whole biological system (human organism), as proposed by V. B. Grinevich. For example, based on his method, V. Grinevich can determine in what cases the opening of ulcer might be generally positive for the organism, since such opening balances the homeostasis of the biological system thus preventing more serious diseases, like myocardial infarction. None of the state-of-the-art methods. for medication assessment permits to make such a conclusion. However, the method proposed by V. Grinevich (with allowance made for the homeostasis of the entire biological system) emphasizes the dynamics and duration of medicinal treatment, rather than its final result, because it is important that the ulcerous defect be not simply healed; but be healed only by the time when the organism completes the dynamic restoration of its homeostasis, i.e., it is important that the ulcerous defect be repaired by the time when the homeostasis of the organism is restored as fully as possible

For example, cases exist when it is recommended to maintain the process of ulcer healing at a slow rate that is safe for the patient, rather than to accelerate the healing too much. This why the period from the beginning of relapse (the first visit to a doctor) till the moment the patient is included into an experimental group is the essentially important criterion in the proposed method; the second essential “inclusion-exclusion” criterion of the new method being the period from the moment the patient is included into the group till the nearest check point. As to previously known assessment methods, they specify the above time periods based on values averaged for a specific population and for a hypothetic progress of disease (for example, ulcer). In other words, all previously know methods depart form the average statistical data typical for healthy biological organisms, which are diffused and non-specific when applied to a specific individual. Therefore, Scheiman's method cannot comprise the essential features of claim 1 through 4, since the claimed method permits to analyze the efficiency of medicinal effect with due regard for the homeostasis of the entire biological system based on individual indications of each of the patients who was included into the experimental group with a certain tolerance of “inclusion-exclusion” criteria. Thus, the Scheiman's method that enables monitoring of the homeostasis leaves out of consideration the interaction between a specific pathology of a biological system and the homeostasis normalization process, which is significant for the assessment of the safety of medicinal treatment.

However, the method proposed by V. Grinevich permits to assess the safety of medicinal treatment by monitoring the homeostasis with due regard for the values of hematological, biochemical and other indications that characterize the safety of the progress of healing by considering appropriate time parameters that are no less important than absolute indications characterizing the homeostasis. The point is, it is important not only to cure the pathology but to cure it in such a time period that permits the biological organism to restore its homeostasis, other wise another, more serious pathology might occur. This is what constitutes, among other, the assessment of the safety of medicinal treatment by the claimed method.

The safety and efficiency of a medication is performed in three phases: the first phase, to put it simply, assesses primarily the safety of the medication, the second one assesses primarily its efficiency, while the third one confirms the results of the two preceding phases. These rules, however, can be strictly applied only to a newly developed medication that is only coming into a wide medical use. As to different mediations that are already widely used by doctors and patients, the above rules permit to compare them only on the basis of absolute values of their efficiency and safety, which show how fast the pathology is cured and whether or not the medication produces undesirable side effects. The method proposed by V. Grinevich can be used at any of the above phases for performing both the efficiency assessment of new medications and the efficiency comparison of medication that are in wide use. Here, the efficiency of a medication implies rather the balance of the pathology healing process and homeostasis restoration process for a specific treated individual, than the effect of rapid curing of a given pathology or absence/presence of side effects. Therefore, the allowance for the interaction of the above processes (pathology curing process and homeostasis restoration process) in the course of assessment of medicinal treatment is unobvious in the monitoring method proposed by Hon et al. The method proposed by V. B. Grinevich permits both to assess the efficiency of new medications and rank the medications that are already in wide use by correlating the above two processes. In case the claimed method is applied for the ranking of widely used medications, it actually establishes the fourth assessment phase, during which a specific medication spreads across the medical market and positions itself among other medications. This means that widely used medications may be re-ranked which will prevent the occurrence of negative effects caused by their use, whereas in the current practice the appropriate U.S administration agencies have to prohibit the use of medications that have already passed the assessment by the state-of-the-art methods only after negative consequences of their use actually occurred. So neither the Redminton's rules, nor the Hon's monitoring method allow assessing the efficiency of medications with a sufficiently reliable statistically validity relative to the correlation of pathology curing process and homeostasis restoration process for a specific patient which is the main factor of the efficiency and safety of an assessed medication.

The above description is considered that of the preferred embodiments only. Modifications of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

1. A method for evaluating the efficiency of a therapeutic action upon a human organism as a function of sex, age, therapeutic action, and characteristics of homeostasis, comprising:
assigning patients to one of a plurality of reference groups, the number of reference groups and the number of patients assigned to each reference group being sufficient to ensure a statistically reliable evaluation of the efficiency of a therapeutic action based on the sex, age, disease characteristics, characteristics of homeostasis, and the time interval from a beginning of disease relapse until therapeutic action is initiated, wherein the results of the therapeutic action are evaluated based on the duration of healing, of the elimination and/or prevention of a pathological sign and by the effect of the therapeutic action on the conditions of homeostasis.

2. The method of claims 1, wherein all patients assigned to a particular reference group differ in age by no more than five years.

3. The method of claims 1, wherein all patients assigned to a particular reference group differ in age by no more than one year.

4. The method of claim 1, wherein the conditions of homeostasis include cholesterol level, high density lipoprotein level, low density lipoprotein level, very low density lipoprotein level, triglyceride level, and atherogenicity factor.

5. The method of claim 1, wherein the results of the therapeutic action are evaluated based on elimination of ulcerous defects, and patients are categorized and assigned to reference groups based on localization of ulcerous defects.

6. The methods of claim 5, wherein the localization of ulcerous defect is categorized into nine different stomach is sections, including cardia, subcardia, upper, middle and lower thirds of the stomach body, angle, anthral and prepyloric section and pylorus.
